(19) United States
(12) Patent Application Publication
(10) Pub. No.: US 2009/0248121 A1
(43) Pub. Date: Oct. 1, 2009

(54) Multi-utility breast thermal therapy delivery system

(76) Inventors: Emmanuelle Miller, Bothell, WA (US); Richard A. Miller, Bothell, WA (US)

Correspondence Address: Emmanuelle Miller, 17403 106th PL NE, Bothell, WA, 98011, US

(21) Appl. No.: 12/322,629

(22) Filed: Feb. 6, 2009

Related U.S. Application Data
(60) Provisional application No. 61/072,583, filed on Apr. 1, 2008.

(51) Int. Cl. A61F 7/02

(52) U.S. Cl. 607/108

(57) ABSTRACT

The invention comprises a thermal therapy delivery device for the entire breast and surrounding lactating tissue, including the armpit area, adaptable for delivering thermal therapy to all impacted tissue with either hot or cold therapy. The device also delivers thermal therapy simultaneously to the areas within and surrounding armpit areas and lactation glands and ducts. Other areas treatable simultaneously include radial zones such as the nipple, areola, surrounding glands, lobules, ducts and connective tissue. To accomplish the thermal delivery objectives, the device main components are delineated in zones comprised of individual cells. Each zone treats a distinct anatomical area, and each zone-addressable component has a specific geometry required to effectively deliver thermal therapy to the entire anatomy of the breast.

### RELATED APPLICATIONS

The present application is a continuation application of U.S. provisional patent application, Ser. No. 61/072583, filed Apr. 1, 2008, for THERMAL DELIVERY PACK FOR THE ENTIRE BREAST TISSUE, by Emma Miller and Richard Miller, included by reference herein and for which benefit of the priority date is hereby claimed.

### FIELD OF THE INVENTION

The present invention relates to a thermal therapy delivery device for the entire breast area and, more particularly, to a therapy device that has many of the advantages of ice packs for the breast with additional novel features that result in a new device capable of delivering simultaneous thermal therapy to the entire breast and adjacent glandular areas with minimal discomfort at high or low temperatures.

### BACKGROUND OF THE INVENTION

The main problem with conventional therapy for the breast is that delivery of thermal therapy to the entire breast tissue simultaneously is not possible. Mammary gland therapy required in engorgement or breast feeding requires entire breast treatment delivered simultaneously, not just the nipple and/or areola. Another problem with conventional breast thermal therapy is the difficulty to interface the thermal storage device with breast contours to insure adequate heat transfer to impacted breast tissue and surrounding glands in the armpit area. Compounding the problem is the fact that these glandular areas also vary not only with cup size but individually as well during engorgement and non-engorged conditions. As a result, products that remain hard, rigid, one constant size, and completely inflexible when thermally charged are not ideal for thermal therapy needs considering breast topography, individual shape variations, and size. Also, conventional ice packs for the breast are not designed to deliver hot or warm thermal therapies that are charged in multiple ways, such as hot water, microwave or refrigeration.

It can be appreciated that ice packs for the breast have been in use for years. Typically, ice packs for the breast are comprised of cooling inserts that are round and sized for the nipple and surrounding areola only. Other ice packs are made for other body parts and are of such inappropriate size and shape which warrants their de-selection as an option for breast therapy. Currently, women may be instructed by their caregivers to utilize frozen peas, ice in a plastic bag, or cabbage leaves to deliver therapy or standard heating pads to deliver hot or warm therapy which are also inadequate both in size and shape to enable their effective use. Women instead resort to hot/warm showers or baths. Other products that have a cover or bra for the cooling inserts have the same design and roughly the same size that result in therapy delivered to the nipple and/or areola only. Existing products made for breast thermal therapy only deliver cooling therapy to the nipple and surrounding areola and are approximately four inches in diameter. Once frozen, these products remain inflexible, only adding to the pain and discomfort when placed on the breast in a medical condition known as engorgement and/or swelling/irritation/bleeding associated with breast feeding.

While these conventional devices may be suitable for the particular purpose to which they address, they are not suitable for delivering simultaneous thermal therapy that can be either hot or cold the entire breast and glandular areas. Areas of particular interest include the tissue within and surrounding the armpit area that also have lactating glands and ducts. The main problem with conventional ice packs for the breast is that delivery of thermal therapy to the entire breast tissue simultaneously is not possible. Mammary gland therapy required in engorgement of breast feeding requires entire breast treatment delivered simultaneously, not just to the nipple and/or areola. Another shortcoming of other solutions is that breast therapy devices currently in use are not concave, do not fit breast contours and do not come in cup sizes to deliver thermal gland therapy for the entire breast and surrounding glands in the armpit area. These devices are not made to accommodate the varying sizes of breasts that occur with breastfeeding and or engorgement in the cessation of lactation. Existing products remain hard and rigid when thermally charged for cooling therapy. This condition is not ideal for thermal therapy needs concerning breast topography and shape. Also, conventional breast therapy devices are not designed to deliver either hot or warm thermal therapies, do not have surfaces that can be effectively cleaned with a damp cloth, and typically are not chargeable with the desired thermal therapy by more than one means.

It would be advantageous to provide a thermal therapy device that includes cold to warm therapies to treat breast engorgement for breastfeeding mothers.

It would also be advantageous to provide a device capable of simultaneously delivering therapy to the entire breast areas.

It would further be advantageous to provide a device capable of simultaneously delivering therapy to tissue within, surrounding the armpit area that too has lactating glands and ducts.

It would further be advantageous to provide a device capable of fitting universal breast sizes to deliver thermal gland therapy that also accommodates the wearers fluctuating breast size.

It would further be advantageous to provide a device capable of charging thermally and remaining flexible enough so that it can be physically wrapped around the breast in all thermal states.

It would further be advantageous to provide a device capable of being charged thermally in cold or warm water as well as the microwave.

It would further be advantageous to provide a device capable of being washed in soap and water.

### SUMMARY OF THE INVENTION

In accordance with the present invention, there is provided an apparatus primarily developed for the purpose of delivering thermal therapy that can be either hot or cold, simultaneously delivering therapy to the entire breast and glands which include tissue within and surrounding the armpit area that contains lactating glands and ducts.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a perspective view of a device showing all components of the thermal therapy delivery system;

FIG. 2 is a top view of a breast thermal therapy delivery device;

FIG. 3 is a rear view of a breast thermal therapy delivery device;

FIG. 4 is a right view of a breast thermal therapy delivery zone;

FIG. 5 is a rear detail view of a breast thermal therapy delivery zone shown without seams and cells;

FIG. 6 is a top perspective view of a breast thermal therapy delivery zone;

FIG. 7 is a top detail view of a d cup specification of a breast thermal therapy delivery device example;

FIG. 8 is a detail view of a device viewed flat and from the top as viewed by the wearer;

FIG. 9 is a detail view of a device viewed flat and as worn from the sides; and

FIG. 10 is a front sectional view of a skin to thermal storage container interface, inside lining and fill cross sectional.

For purposes of clarity and brevity, like elements and components will bear the same designations and numbering throughout the Figures.

### DESCRIPTION OF THE PREFERRED EMBODIMENT

The invention consists of a concave, flexible, pliable thermal storage container in cells containing a thermal storage material 14. The device is shaped to fit over the entire breast and effectively deliver a range of thermal therapy from hot or warm to cold or frozen to the glands within the breast and surrounding tissues also located in the armpit region. Treatable regions for the device include lactating glands, lobules, nipple, areolas, ducts, and connective tissue including the armpit region.

The device incorporates a cup thermal storage container 22 shape, far more effective in relieving pain and discomfort by simultaneously delivering thermal therapy to the entire breast and surrounding tissue to address swelling or other discomfort associated with lactation complications.

The concave cup structure incorporates a winged edge underarm thermal storage container 18 that extends into the underarm region to deliver cold or heat therapy to glands, ducts, connective tissue within the arm pit area.

The device also incorporates a nipple flap thermal storage container 20 that may or may not be utilized as it can be folded under the device if nipple therapy is not desired, or placed on the nipple/areola area if thermal therapy is desired in this region.

The device extends to accommodate sizes from AA, A, B, C, and/or D, in the small size and D, E, EE, with the larger size to provide a thermal delivery pack for the entire breast tissue and offer a size choice in a cup so that women can customize the delivery area for their specific thermal need.

The thermal storage material 14 can be any gel or other non-solid thermal storage media that is non-toxic and has the necessary heat transfer characteristics. Thermal storage material 14 can be moved around to different regions of the container so as to accommodate changing breast size. The flap can be folded under once gel is squeezed out of the leading edge of the device.

The thermal storage container-to-skin interface 16 is the interface between the thermal storage container and the body part to be treated. The device is positioned and held to body part either manually, by hooks, loop, or by clothing such as a sleep or sport bra. Alternatively, the device can slide into a sheath or have a barrier against direct skin contact to insulate skin from excessive heat or cold if a higher thermal charge is preferred.

A slit in the thermal container offers an adjustable size option for the engorged or non-engorged breast to accommodate change in breast size during lactation.

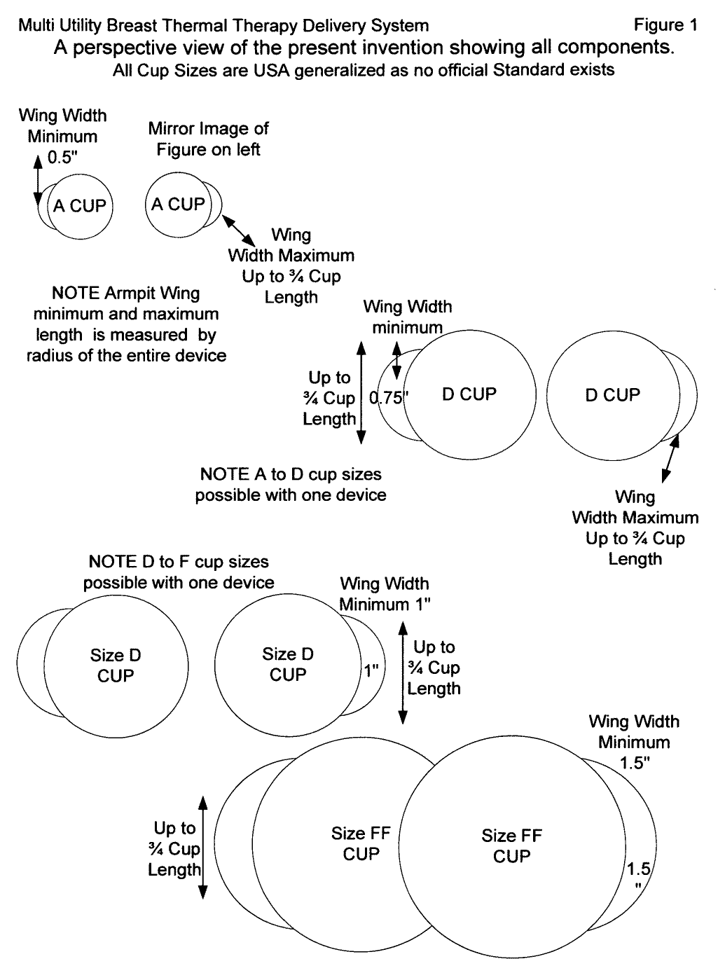

FIG. 1 is a perspective view of a device showing all components of the thermal therapy delivery system.

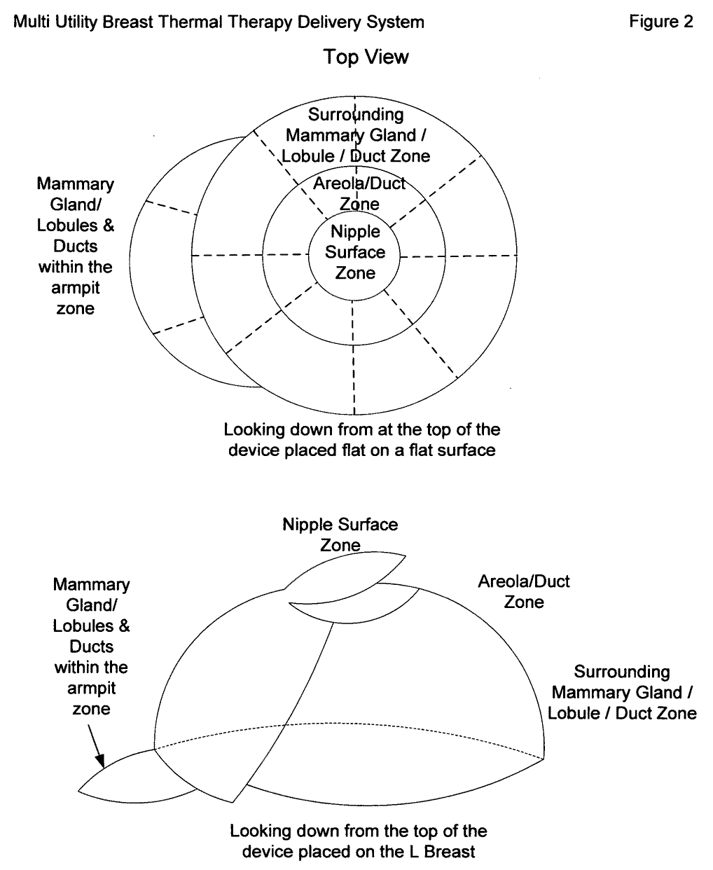

FIG. 2 is a top view of a breast thermal therapy delivery device.

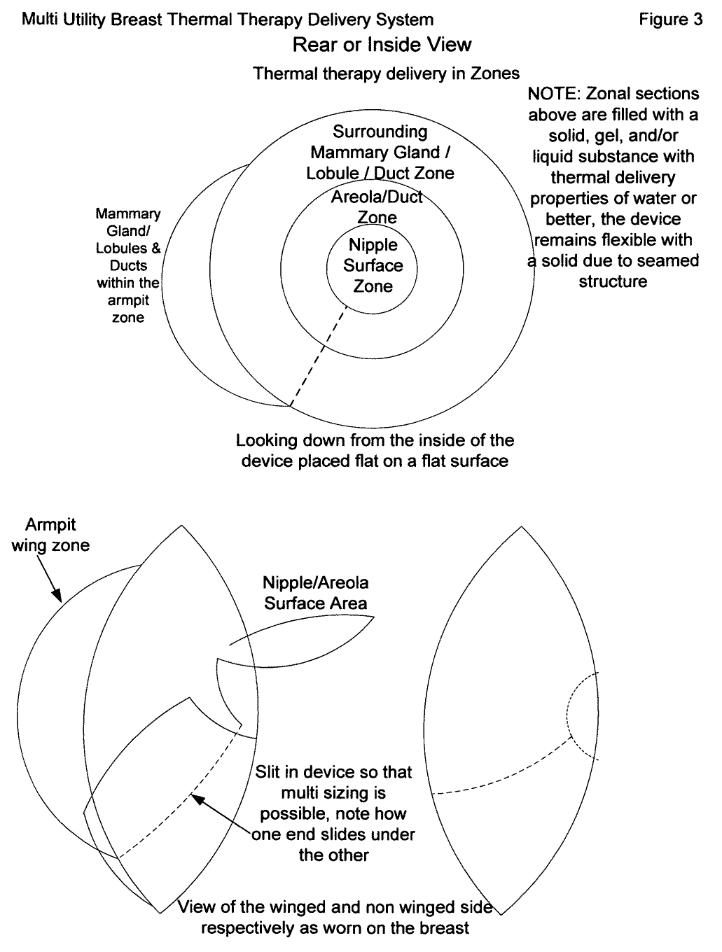

FIG. 3 is a rear view of a breast thermal therapy delivery device.

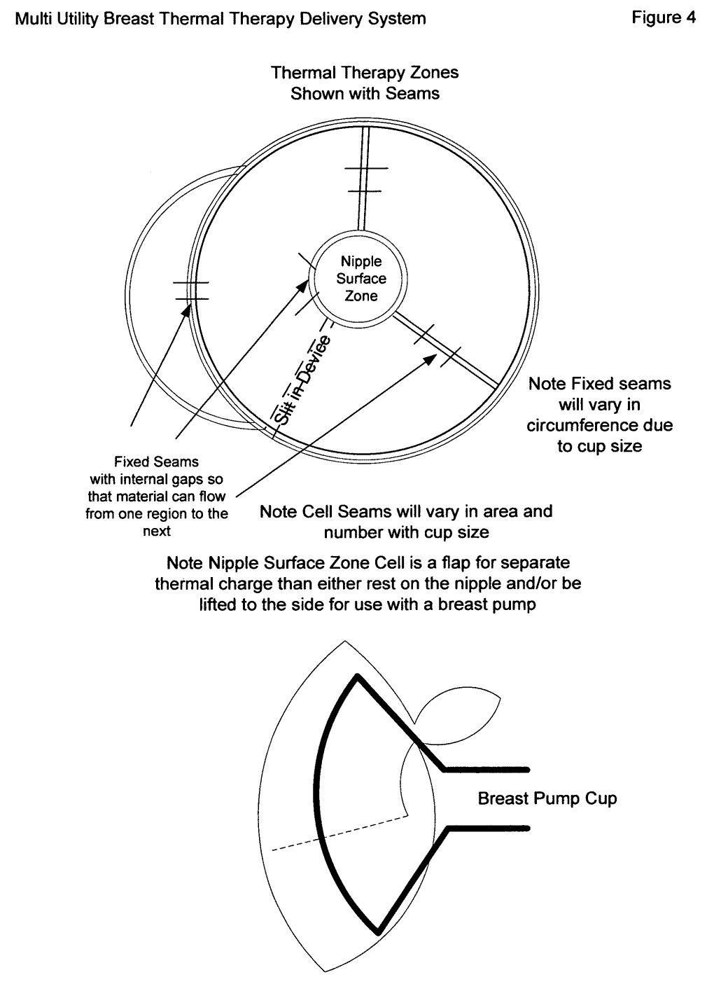

FIG. 4 is a right view of a breast thermal therapy delivery zone.

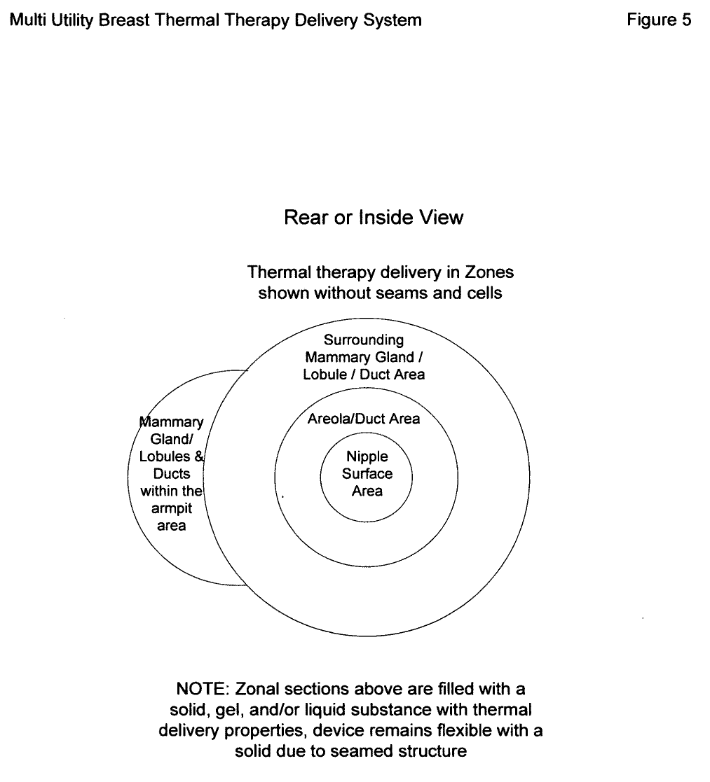

FIG. 5 is a rear detail view of a breast thermal therapy delivery zone shown without seams and cells.

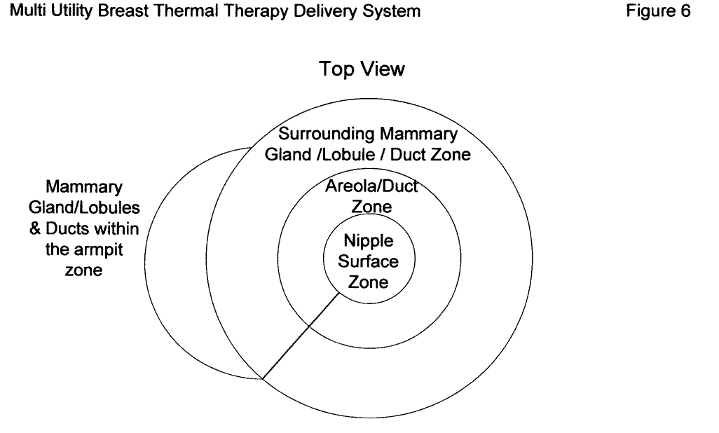

FIG. 6 is a top perspective view of a breast thermal therapy delivery zone.

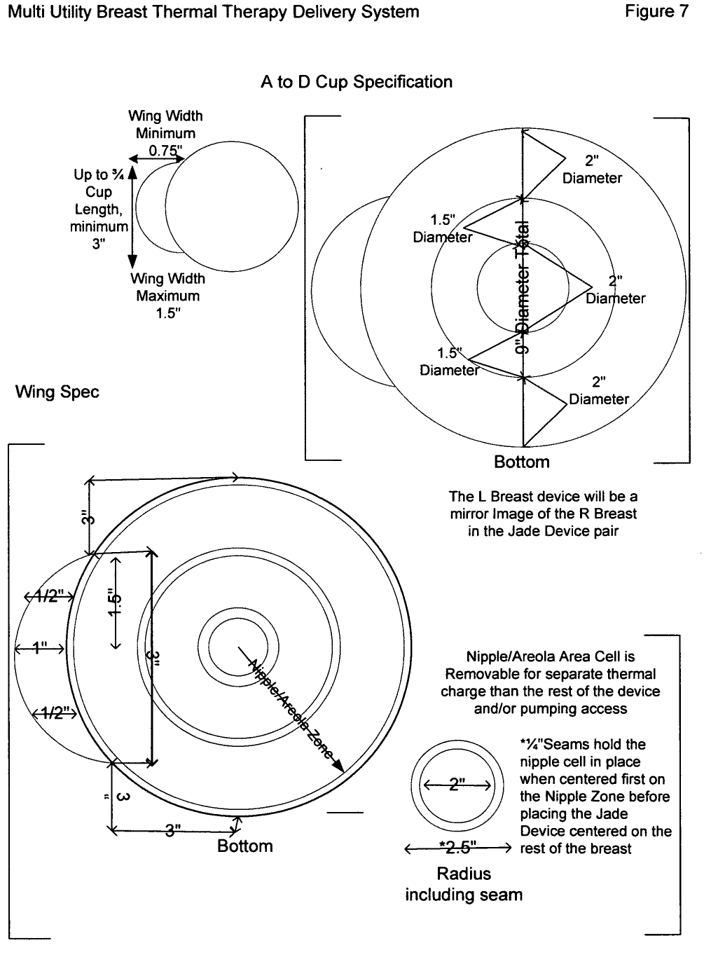

FIG. 7 is a top detail view of a d cup specification of a breast thermal therapy delivery device example.

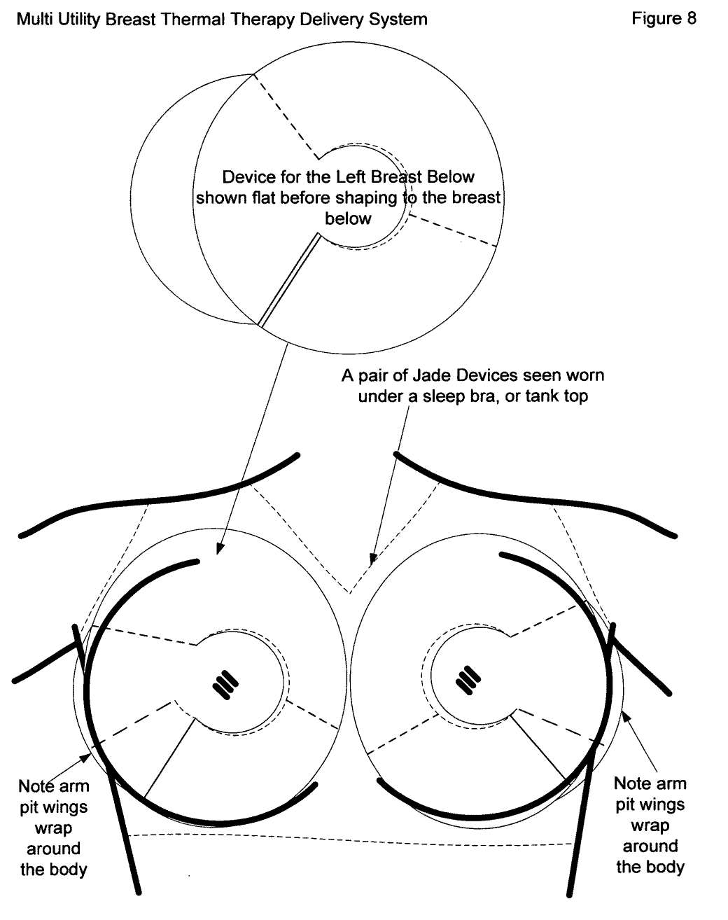

FIG. 8 is a device view from the top as flat and as worn from the perspective of the wearer.

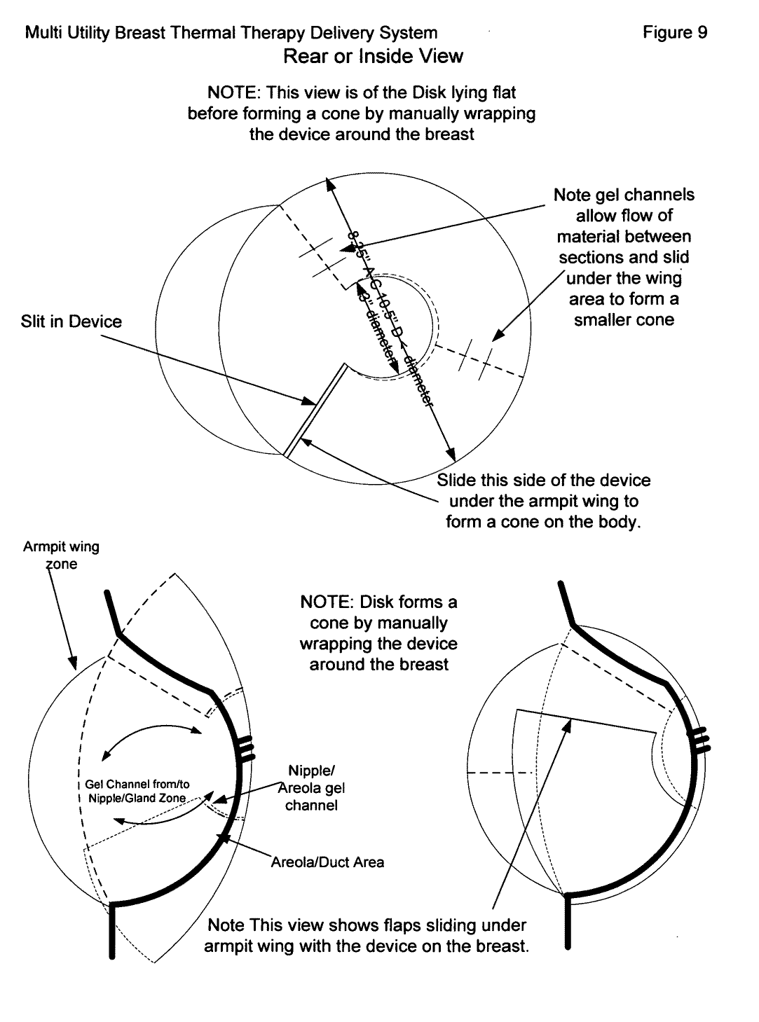

FIG. 9 is a device viewed as flat and as worn from the sides.

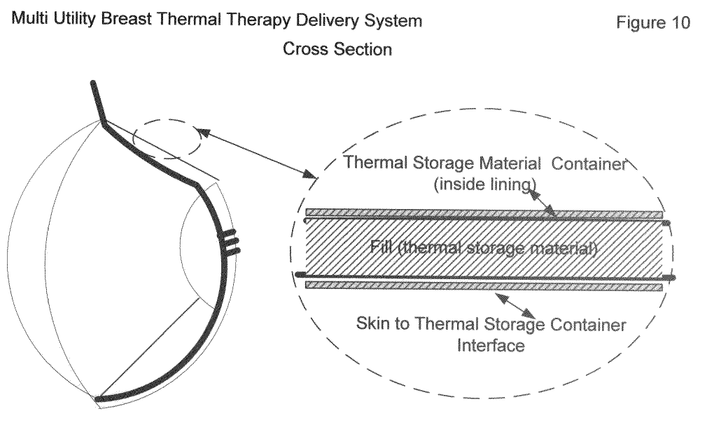

FIG. 10 is a device viewed from the front with a cross section of the skin to thermal storage container interface, inside lining and fill cross section.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A multi-utility breast thermal therapy delivery system for delivering thermal therapy to the entire breast and surrounding tissue, comprising:
means for storing the desired thermal state;
means for maintaining skin-tolerable thermal conditions at all thermal states;
means for containing the thermal storage material in the underarm areas, structurally bonded to said means for maintaining skin-tolerable thermal conditions at all thermal states, and sealably inserted to said means for storing the desired thermal state;
means for containing the nipple flap thermal storage material, structurally bonded to said means for maintaining skin-tolerable thermal conditions at all thermal states, and sealably inserted to said means for storing the desired thermal state; and
means for containing the thermal storage material in the cup area, structurally bonded to said means for maintaining skin-tolerable thermal conditions at all thermal states, and sealably inserted to said means for storing the desired thermal state.

2. The multi-utility breast thermal therapy delivery system in accordance with claim 1, wherein said means for storing the desired thermal state comprises a material with specific heat of water or greater, remains flexible in all thermal states, hold thermal charge for at least 4 minutes, non-toxic material, flowable, re-usable thermal storage material.

3. The multi-utility breast thermal therapy delivery system in accordance with claim 1, wherein said means for maintaining skin-tolerable thermal conditions at all thermal states comprises a non-toxic, non-wettable, flexible, adequate heat transfer rate, moldable to body contour, skin tolerable at all thermal states, re-usable thermal storage container-to-skin interface.

4. The multi-utility breast thermal therapy delivery system in accordance with claim 1, wherein said means for containing the thermal storage material in the underarm areas comprises a shapable, re-usable, non-toxic, adequate heat transfer rate, flexible at all thermally charged states, winged edge underarm extention underarm thermal storage container.

5. The multi-utility breast thermal therapy delivery system in accordance with claim 1, wherein said means for containing the nipple flap thermal storage material comprises a flexible at all thermally charged states, non-toxic, re-usable, shapable, adequate heat transfer rates, nipple flap extention nipple flap thermal storage container.

6. The multi-utility breast thermal therapy delivery system in accordance with claim 1, wherein said means for containing the thermal storage material in the cup area comprises a flexible at all thermally charged states, non-toxic, re-usable, adequate heat transfer rate, concave cup shapable, extendable size cup thermal storage container.
